(19) United States
(12) Patent Application Publication
(10) Pub. No.: US 2018/0014996 A1
(43) Pub. Date: Jan. 18, 2018

(54) SELF SHEATHING ANESTHETIC NEEDLE WITH A DEDICATED SYRINGE

(71) Applicant: HBA MEDICAL GROUP, INC., San Diego, CA (US)

(72) Inventor: Hooman Asbaghi, San Diego, CA (US)

(21) Appl. No.: 15/207,806

(22) Filed: Jul. 12, 2016

(51) Int. Cl. A61J 1/20; A61M 5/32; A61M 5/31

(52) U.S. Cl. CPC A61J 1/2006; A61M 5/3243; A61M 5/3205; A61M 5/3137; A61M 2210/0625; A61M 2210/0631

(57) ABSTRACT

A self-sheathing injection assembly that includes a sheath protecting the medical professional from accidental punctures from the needle. The sheath is slidable to expose the end of the needle, and the sheath is lockable in the exposed condition with a detent mechanism. The separable needle assembly also includes features that firmly couples a carpule of medicine to the needle assembly, such that upon removal of the needle assembly after use the carpule remains attached to the needle for simultaneous disposal.

### FIELD OF THE INVENTION

Various embodiments of the present invention pertain to a safety syringe for providing medicaments to patients, and in particular to safety syringes for use with dental patients.

### BACKGROUND OF THE INVENTION

The American Dental Association (ADA) attributes most needlestick and subsequent infections in dental offices to the handling of sharp instruments and needles which must be handled carefully to prevent injuries and recapped after use. If a patient requires multiple injections from a single syringe, the needle should be recapped between each use to preclude the possibility of needlestick injury. At best, needles may be recapped by placing the cap in a special holder, by using forceps or other appropriate instrument to grasp the cap, or by simply laying the cap on the instrument tray and then manually guiding the needle into the cap until it is completely sealed. The greatest risk of needlestick injury occurs when the cap must be manually placed over the used needle. Almost 90% of needlestick injuries occur either when the operator withdraws the needle from the patient, or between injections. Operators usually do not bother to take the time to re-cap the needle between injections, and leave the bare infected needle uncovered on the tray, and this is precisely when accidents occur.

The lack of passive devices specifically designed for injury prevention has shown itself to be one of the greatest obstacles in preventing operator exposure to infected blood and bodily fluids. According to past clinical reports, dental students and dental assistants had the highest rate of exposure; syringe needle injuries were the most common type of exposure; and giving injections, cleaning instruments after procedures and drilling were the activities most frequently associated with exposure. According to another past study, 1 out of 6 needlestick injuries to New York City healthcare workers occurred among dental workers, and 10%-18% of these latter injuries resulted in HB infection.

What is needed are apparatus and methods that provide increased safety to medical professionals when they deliver medicine to a patient. Various embodiments of the present invention do this in novel and unobvious ways.

### SUMMARY OF THE INVENTION

One aspect of the present invention pertains to a method for dispensing medicine from a container with a syringe. Some embodiments include providing a syringe with compartment, a pump, and a sliding finger hold, and a separable needle assembly including a slidable sheath surrounding the sharp tip. Other embodiments include placing the container in the compartment. Yet other embodiments include attaching the needle assembly to the syringe after the placing. Still other embodiments include engaging the sheath with the finger hold. Yet other embodiments include sliding the finger hold and exposing the sharp tip, locking the finger hold in position with the sharp tip exposed, pumping medicine through the attached needle, and applying a force with the finger hold to unlock from the position.

Another aspect of the present invention pertains to a method for dispensing medicine from a container with a syringe. Some embodiments include providing a syringe with compartment and a pump, and a separable needle assembly including a container-holding receptacle. Other embodiments include placing the container in the compartment. Still other embodiments include attaching the needle assembly to the syringe after the placing. Yet other embodiments include locating the container in the receptacle by the attaching, pumping medicine through the attached needle, removing the needle assembly from the syringe, and retaining the container in the receptacle during and after the removing

Yet another aspect of the present invention pertains to an assembly for dispensing liquid from a container. Some embodiments include a syringe assembly having a distal end with an opening to a chamber, the opening being adapted and configured to receive a container of the liquid therethrough, the chamber being adapted and configured to hold the container, the syringe including a finger-operated pump for expelling liquid from the container. Yet other embodiments include a separate needle assembly including a needle with a sharp tip, a sheath surrounding the sharp tip and slidable from a position surrounding the sharp tip to a position exposing the sharp tip, a receptacle with an opening for receiving therein an end of the container, the receptacle including a puncturing projection located within the receptacle, the needle assembly being adapted and configured to releasably couple to the syringe assembly, and means for retaining the container in the receptacle during removal of the needle assembly from the syringe assembly, wherein coupling the needle assembly to the syringe assembly punctures the end of the container by the projection.

Still another aspect of the present invention pertains to an assembly for dispensing liquid from a container. Some embodiments include a syringe assembly having a distal end with an opening to a chamber, the opening being adapted and configured to receive a container of the liquid therethrough, the chamber being adapted and configured to hold the container, the syringe including a finger-operated pump for pressurizing the liquid and an arm slidable between a first position and a second position, the arm and the syringe being adapted and configured to releasably lock the arm on the syringe assembly in the second position. Other embodiments include a separate needle assembly including a needle with a sharp tip and a lumen, a sheath surrounding the sharp tip and slidable from a position surrounding the sharp tip to the second position exposing the sharp tip, a receptacle adapted and configured for receiving therein an end of the container, and a puncturing projection located within the receptacle, wherein coupling the receptacle to the end of the container punctures the end of container by the projection, and sliding the arm to the second position moves the sheath to the second position.

It will be appreciated that the various apparatus and methods described in this summary section, as well as elsewhere in this application, can be expressed as a large number of different combinations and subcombinations. All such useful, novel, and inventive combinations and subcombinations are contemplated herein, it being recognized that the explicit expression of each of these combinations is unnecessary.

### BRIEF DESCRIPTION OF THE DRAWINGS

Some of the figures shown herein may include dimensions. Further, some of the figures shown herein may have been created from scaled drawings or from photographs that are scalable. It is understood that such dimensions, or the relative scaling within a figure, are by way of example, and not to be construed as limiting.

FIG. 1 is a side perspective computer generated photographic representation of a safety syringe assembly according to one embodiment of the present invention.

FIG. 2A is a perspective CAD line drawing of a portion of the apparatus of FIG.

FIG. 2B is a side elevational computer generated photographic representation of a portion of the apparatus of FIG. 1.

FIG. 3A is a computer-generated photographic representation of a perspective end view of the apparatus of FIG. 2B.

FIG. 3B is a side perspective view of the apparatus of FIG. 3A.

FIG. 3C is a side perspective view of the other side of the apparatus of FIG. 3B.

FIG. 4A is a perspective, exploded CAD line drawing of the apparatus of FIG. 2A.

FIG. 4B is a side elevational CAD line drawing of the apparatus of FIG. 2A.

FIG. 4C is a view from the top, looking down on the apparatus of FIG. 4A.

FIG. 5A is a cross-sectional view of the apparatus of FIG. 4A with the detent mechanism shown in the storage position.

FIG. 5B is a view of the apparatus of FIG. 5A with a carpule inserted.

FIG. 5C is a view of the apparatus of FIG. 5A except with the detent mechanism shown in the temporary position.

FIG. 6A is a computer-generated photographic representation of the apparatus of FIG. 2A showing a carpule being inserted.

FIG. 6B is a view of the apparatus of FIG. 6A showing the apparatus of FIG. 2B being attached.

FIG. 7A is a computer-generated perspective photographic representation of the distal end of the apparatus of FIG. 5B, with portions of the needle body being shown as semi-transparent.

FIG. 7B is a view of the apparatus of FIG. 7A showing the apparatus of FIG. 2B brought into close proximity to the distal end of the carpule, with portions of the needle body being shown as semi-transparent.

FIG. 7C is a representation of the apparatus of FIG. 7B with the apparatus of FIG. 2b shown in the position as in FIG. 6B, with portions of the needle body being shown as semi-transparent.

FIG. 8A is a side elevational computer-generated photographic representation of the apparatus of FIG. 7B.

FIG. 8B is a side elevational view of the apparatus of FIG. 8A with the needle assembly shown in the locked position.

FIG. 8C is a view of the apparatus of FIG. 8b with surface shading removed and showing internal components.

FIG. 9A is a side elevational view of the apparatus of FIG. 8B, except from the other side.

FIG. 9B is a close-up representation of the apparatus of FIG. 9A with the detent mechanism being located between the safe, storage position and the exposed, temporary position.

FIG. 9C is a view of the apparatus of FIG. 9A with the detent mechanism shown in the fully exposed, temporary position.

FIG. 10A is a view of the apparatus of FIG. 9C prior to insertion of the distal end of the needle into the patient's gum.

FIG. 10B is a perspective view of the apparatus of FIG. 10A with the needle inserted and the syringe spring compressed.

FIG. 11 is a view of the apparatus of FIG. 9A after use, with the syringe and carpule being removed as a unit.

### ELEMENT NUMBERING

The following is a list of element numbers and at least one noun used to describe that element. It is understood that none of the embodiments disclosed herein are limited to these nouns, and these element numbers can further include other words that would be understood by a person of ordinary skill reading and reviewing this disclosure in its entirety.
20 injection assembly
24 carpule
25 distal end
30 syringe assembly
34 body
35 finger hold
36 spacer
37 spring
38 cap
39 guide
40 shoulder
44 holder
45 channel
46 entrance
47 port
48 chamber
50 detent assembly
51 finger hold
52 claw, projection
53 rail
54 shoulder
56 latch
60 pump
61 finger hold
62 shaft
63 harpoon
80 needle assembly
82 body
83 channel
84 spring
85 connector
86 sliding block
87 housing
88 receptacle
89 slot
90 retaining ring
91 ears
92 compartment
93 B bevel
94 cavity
96 sheath
98 needle
99 A bevel

### DETAILED DESCRIPTION OF ONE OR MORE EMBODIMENTS

For the purposes of promoting an understanding of the principles of the invention, reference will now be made to the embodiments illustrated in the drawings and specific language will be used to describe the same. It will nevertheless be understood that no limitation of the scope of the invention is thereby intended, such alterations and further modifications in the illustrated device, and such further applications of the principles of the invention as illustrated therein being contemplated as would normally occur to one skilled in the art to which the invention relates. At least one embodiment of the present invention will be described and shown, and this application may show and/or describe other embodiments of the present invention, and further permits the reasonable and logical inference of still other embodiments as would be understood by persons of ordinary skill in the art.

It is understood that any reference to “the invention” is a reference to an embodiment of a family of inventions, with no single embodiment including an apparatus, process, or composition that should be included in all embodiments, unless otherwise stated. Further, although there may be discussion with regards to “advantages” provided by some embodiments of the present invention, it is understood that yet other embodiments may not include those same advantages, or may include yet different advantages. Any advantages described herein are not to be construed as limiting to any of the claims. The usage of words indicating preference, such as “preferably,” refers to features and aspects that are present in at least one embodiment, but which are optional for some embodiments, it therefore being understood that use of the word “preferably” implies the term “optional.”

This document may use different words to describe the same element number, or to refer to an element number. It is understood that such multiple usage is not intended to provide a redefinition of any language herein. It is understood that such words demonstrate that the particular feature can be considered in various linguistical ways, such ways not necessarily being additive or exclusive.

Incorporated herein by reference is U.S. Pat. No. 6,530,905, filed Feb. 1, 2001, titled SELF-SHEATHING DENTAL NEEDLE, incorporated herein.

Various embodiments of the present invention pertain to a sheathed needle that provides medicine from a container by way of a syringe assembly. Preferably, the needle assembly, liquid container, and syringe assembly are three separate components that are brought together by the medical professional to provide medicine to a patient. The contents of the liquid container are not limited, and in some medical applications the container includes within it an anesthetic, such as for dentistry, cosmetic procedures, wound care, and the like. However, various other embodiments of the present invention contemplate non-medical applications.

After the medicine is injected, the used needle assembly is removed from the syringe assembly, with the used liquid container (such as a carpule) safely attached to the needle assembly, such that the two components stay together as a unitary assembly, at least for purposes of disposal and routine handling. Therefore, the used needle assembly (with the sheath again covering the sharp tip) is sufficiently connected to the carpule such that the two components are disposed of together.

Although what will be shown and described herein pertain to an injection assembly that is adapted and configured to provide medicine to a patient, it is understood that the present invention contemplates yet other embodiments. As one example, in some industries a liquid such as a lubricant, adhesive, or inspection fluid (such as the type that glows under ultraviolet light) are applied in relatively small amounts, in precise manners, on a component by a user. Various embodiments of the present invention contemplate such non-medicinal uses.

Various embodiments of the present invention further include a detent mechanism by which the user of the injection device can move a protective sheath to a temporary usage position in which the sharp tip is no longer surrounded. A locking mechanism or detent mechanism retains the sheath in this exposed position. Preferably, a combination of a spring force and exterior mechanical surface features readily couple and uncouple together to maintain this exposed usage position, although some embodiments do not utilize a spring force, and instead use a combination of mechanical features (such as a bayonet-type fitting).

Further, although what will be shown and described is operation of an injection assembly that can be accomplished with a single hand, yet other embodiments of the present invention contemplate two hand usage, such as in which a first hand holds the syringe steady while the second hand moves the needle sheath to the detent position. The retention feature and detent feature will now be described in more detail.

One embodiment of the present invention pertains to a needle assembly that includes one or more features for retaining it to the end of a liquid container, such as a carpule used by medical professional. In one embodiment, the needle assembly and container are intended to be disposable after a single use. After the contents of the coupled carpule is injected through the needle, the needle assembly and carpule remain safely coupled together to facilitate efficient disposal of the used needle and the used carpule. By maintaining this coupling, there is a reduction in the possibility of the medical professional or others being punctured by either end of the needle, placed in contact with bodily fluids of the patient, or placed in contact with the medicine.

In some embodiments, this coupling is achieved frictionally. In such embodiments, the needle assembly includes a retaining feature, such as a retaining ring, that readily slides onto the end of the carpule. Relatively low frictional forces are required for this engagement, although this engagement does include the axial force necessary for an end of a hollow needle to puncture the carpule. However, the retaining features are adapted and configured such that any attempt to separate the carpule from the retaining ring requires a larger force. This larger force required for separation is sufficient to overcome the forces pulling back the harpoon end of the pump assembly, and further sufficiently large to overcome any drag forces or typically encountered accidental forces that would otherwise tend to separate the carpule from the needle assembly. Therefore, the carpule and needle assembly are easy to put together, but difficult to take apart. Once the needle assembly and carpule are engaged and retained together, the larger force required to separate the two components is so sufficiently large that with normal handling the two components stay engaged with each other through removal of the needle assembly, and disposal of the jointly coupled needle assembly and carpule.

In some embodiments, the means for retaining the container in the needle assembly receptacle is of a self-energizing nature, in general principle similar to self-energizing brakes on a car. In these embodiments, the axial load applied to remove the carpule creates a proportional frictional load that is equal to the separating load. For that reason, the carpule does not separate. For at least low separation loads (such as that which would accidentally be applied by a medical professional, or the typical kinds of loads that might otherwise be accidentally applied against the carpule) the separating load creates drag on a retention feature. This drag deforms the retention feature (preferably but not necessarily, elastically) such that the deflecting member achieves a higher normal load (i.e., radially applied) against the periphery of the carpule. This higher normal surface load in turn creates a higher frictional load on the surface of the carpule that opposes the separation load. Therefore, within limits, the higher the separation force applied to separate the carpule from the needle assembly, the higher the friction load will be to keep the two engaged together.

Although what has been shown and described is a means for retention that includes frictional coupling, still further embodiments of the present invention include retention means that operate with or without friction, but which further include a hooking or grappling feature in the retaining ring. This hooking feature is adapted and configured to permit smooth, easy insertion of the carpule into the retaining ring. However, the application of a reverse force (a separation force) causes a change in the geometry of the hooking feature such that an end of the feature digs into the surface of the carpule. In such embodiments, the means for retaining therefore includes a feature that mechanically interferes with the removal of the carpule. As one example, a J-shaped hook, having the rounded end of the hook being the first part of the hook to encounter the end of the carpule during insertion, would have a stiffness low enough to permit the hooking feature to simply bend out of the way during insertion. However, the application of a separation force would permit the deformation of the hooking feature (elastically or inelastically), such that it digs into the surface, and the application of still further removal forces would cause increased deformation. In some embodiments, the free end of the hook includes a barb

In yet another embodiment, the retention means incorporates a “Chinese handcuff” feature. In at least some of such embodiments, the retention feature would generally surround the end of the inserted carpule, and would be sufficiently deformable to expand out of the way during insertion. However, any attempt to remove the carpule would cause the retention feature (such as a sock-type feature) to collapse around and snug up against the external surface of the carpule. In some embodiments, this feature has somewhat of a funnel-shaped end to permit easy insertion of the carpule, but an adjacent section of the retaining feature that comes into close contact with the outer diameter of the carpule. Any attempt to remove the carpule causes this diameter to attempt to axially compress the funnel, and this compression causes the funnel to tighten its snugness against the diametral surface of the carpule.

In yet other embodiments, the retention means includes mechanical features that have a shape and stiffness that permit relatively easy insertion of the carpule, but during removal these features convert the drag of the carpule surface against the feature into an increased normal force against the outer diameter, and an increased axial, frictional load on the outer diameter.

Yet other embodiments of the present invention include a locking mechanism that retains the sheath of the needle in a usage position, with the needle exposed. In one embodiment, the syringe assembly includes a flexible rail, sliding member, or arm that can be pulled or pushed from a storage position into a usage position, and in so doing likewise move the sheath from the storage position to the usage position. After the professional has pushed or pulled the sliding member in order to expose the needle tip, a detent feature of the sliding member is placed in a locked position. In some embodiments, this locked position is achieved with a portion of the sliding member that is spring loaded away from a more relaxed position with the sliding member is in the storage position. This spring load can be achieved by flexure of the rail assembly, compression or extension of one or more springs in the syringe assembly, or compression or extension of one or more springs in the needle assembly, or combinations of such springs.

As the sliding member is moved by the professional to the usage position, a pair of surface features (such as one on the sliding member and one syringe body) are able to couple together the body of the syringe assembly and sliding member, such that this coupling occurs with the needle tip exposed and further with changes in some aspect of the spring load. In the usage position, this detent feature can therefore be in a more relaxed state, or in a state providing more spring load. In some embodiments, this spring load is achieved by a first detent surface feature of the rail that extends in a direction orthogonal to the sliding direction, such as a bump, projection, or hook. Therefore, the detent motion by the user includes combined motion in both a sliding direction and also in a direction other than the sliding direction. In order to place this feature into the temporary spring state, one of the features also moves in an orthogonal direction, and can achieve a mechanical interference-type of detent with the second surface feature. This rail cannot be slid back to the storage position, because the two features of the syringe body and rail are in the way of one another, and interfere with this movement. In order to move the sliding arm back to the usage position, the medical professional must separate the detent feature by pushing it or pulling it relative to the body. This motion to overcome the detent is at least partly orthogonal to the sliding movement, consistent with the detent spring force being in a direction orthogonal to the general sliding movement.

In some embodiments the operation of the syringe includes sliding a member in a first direction (such as to expose the sharp tip of the needle), but also moving the sliding member in a second direction different than the sliding direction (such as a linearly orthogonal movement or a rotational movement). This second movement (not fully in the direction of sliding) can provide either an increase in a spring load, or decrease in a spring load, such that a detent feature of the sliding member is moved (linearly or rotationally) to a location in which the detent fixture can rest against a complementary-shaped feature on the syringe body. This shape and complementary shape are preferably locked together by this spring force, such that the two features stay coupled.

In one embodiment, the sliding arm includes a hook-shaped feature, and the syringe body includes a reduced diameter, shoulder feature. The medical professional would move the hook away from the syringe body, followed by an axial movement to place the hook in alignment with the shoulder, and thereafter, while retaining the axial position, relax the spring load to let the hook (or projection) to rest within the reduced-diameter shoulder. It is further understood that such detent operation could also be achieved between a hook and an increased-diameter shoulder, in which case the flexing spring load of the sliding member could be maintained or increased when placed in the usage position.

It is understood that many other types of detent features are contemplated by other embodiments, including as one example a projecting pin and a hole. The pin could be placed on the sliding member (such as replacing the hook shown on the sliding arm herein), and the hole placed in the body of the syringe. Likewise, the pin could be located on the body, and the hole located on the sliding member. In still further embodiments, the pin could be spring loaded, such that the spring pops into location once it is aligned with the hole, and the practitioner disengages the spring loaded pin once the injection is complete. Still further, instead of a pin, the detent feature could be a ball retained in a socket, with or without a spring load applied to the ball within the socket. Numerous other types of currently existing detent mechanisms could be applied to the injection assemblies described herein, either for one handed or two handed operation.

Still further, although what is shown and described in detail herein is a sliding member in which a detent action is achieved by pulling the sliding arm away (out of) the direction of sliding, yet other embodiments contemplate a detent motion that is sideways or lateral to the direction of sliding. Still further embodiments contemplate a detent motion in a combined axial and circumferential direction, such as bayonet-type features.

Further, what is shown and described incudes the finger hold of a rail that is pulled away (out of the groove), and then released back into a reduced diameter groove (i.e., first flexing the sliding member to a state of higher spring load, and then after sliding the member rearwardly allowing the member to flex back to a position of lower spring load), yet other embodiments contemplate single action detent mechanisms. In such embodiments there is a single motion to achieve a detented retention. For example, such embodiments contemplate pulling and permitting the arm to flex toward the syringe body in an interference-type coupling, or simply pulling the sliding member backward until the spring forces by themselves pull the detent feature of the sliding member into coupling with the detent feature of the body. In this second case, the single action required by the medical professional to go from the usage state back to the storage state would be a single action to pull and flex the sliding arm away from the body and back into its sliding channel.

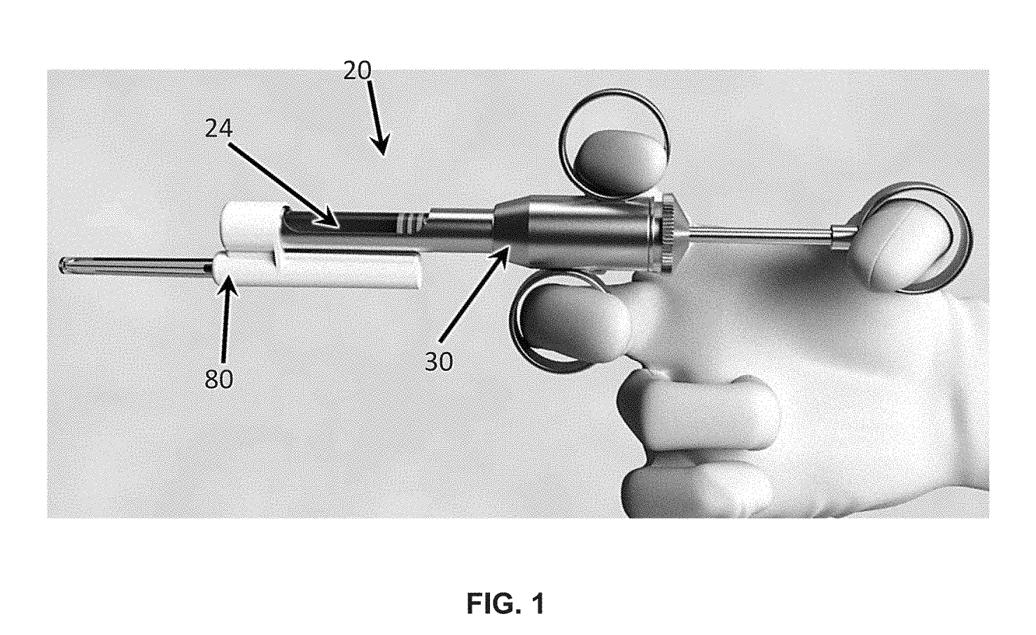

FIG. 1 shows an injection assembly 20 according to one embodiment of the present invention. Assembly 20 includes a syringe assembly 30 that supports a container 24 of medicine in a forward chamber 48. A readily separable needle assembly 80 is located at the distal end of syringe assembly 30, and couples both mechanically and for fluid communication to container 24, and further couples mechanically to the distal end of syringe assembly 30.

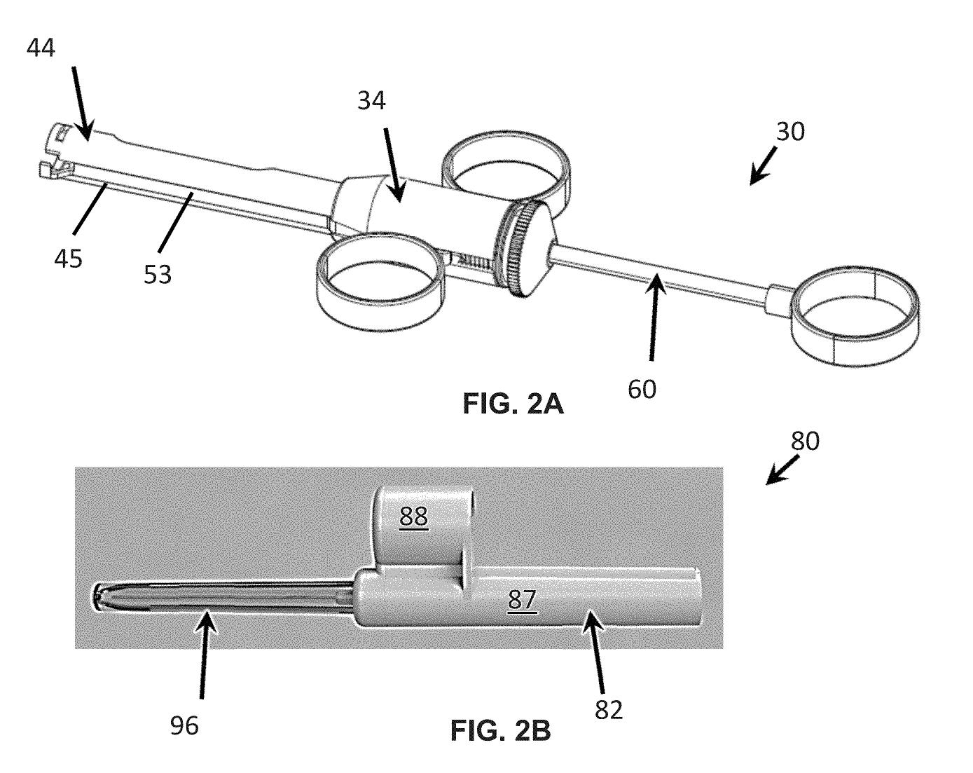

FIGS. 2A and 2B show the syringe assembly 30 and needle assembly 80, respectively. Syringe assembly 30 includes a mechanical pumping assembly 60 located within a body 34. A holder 44 for container 24 is attached to body 34. Referring also to FIG. 1, it can be seen that syringe 30 includes multiple finger holes for permitting a medical practitioner or other user to conveniently and safely operate injection assembly 20 with one hand. In some embodiments, syringe assembly 30 is fabricated from stainless steel. Some medical professionals prefer the weight and feel of an assembly fabricated from steel. However, the present invention contemplates syringe assemblies made from any material, including plastics.

FIGS. 2B, 3A, 3B, 3C, and 8C depict various aspects of needle assembly 80. Assembly 80 includes a body 82 having a top receptacle 88 located adjacent a housing 87. In some embodiments, needle assembly 80 includes a hollow needle that extends from an external A bevel that penetrates the patient to a B bevel that penetrates the cartridge. In various embodiments of the present invention, both the A bevel and B bevel are protected to prevent accidental penetration of the user of the injection assembly. As will be noted in the discussion to follow, the A bevel is surrounded in its storage position by a protective sheath. The B bevel is recessed within its holding structure, such that in normal use any finger of the user is unlikely to touch the B bevel.

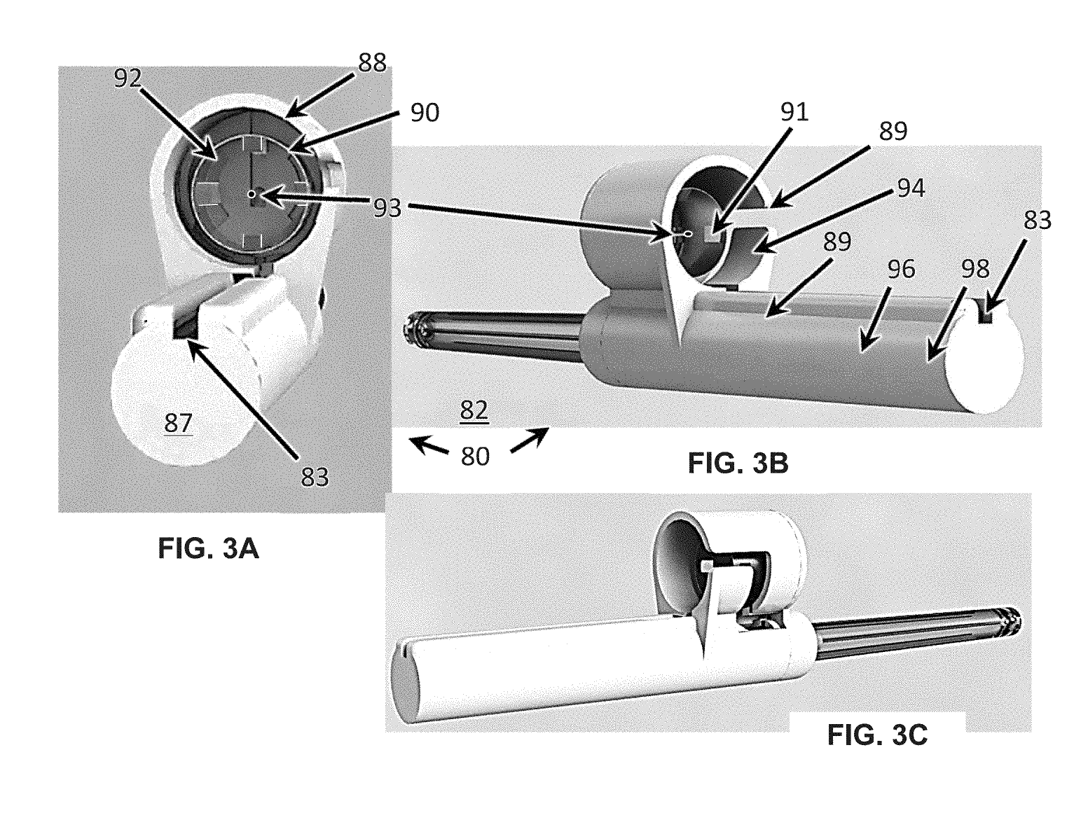

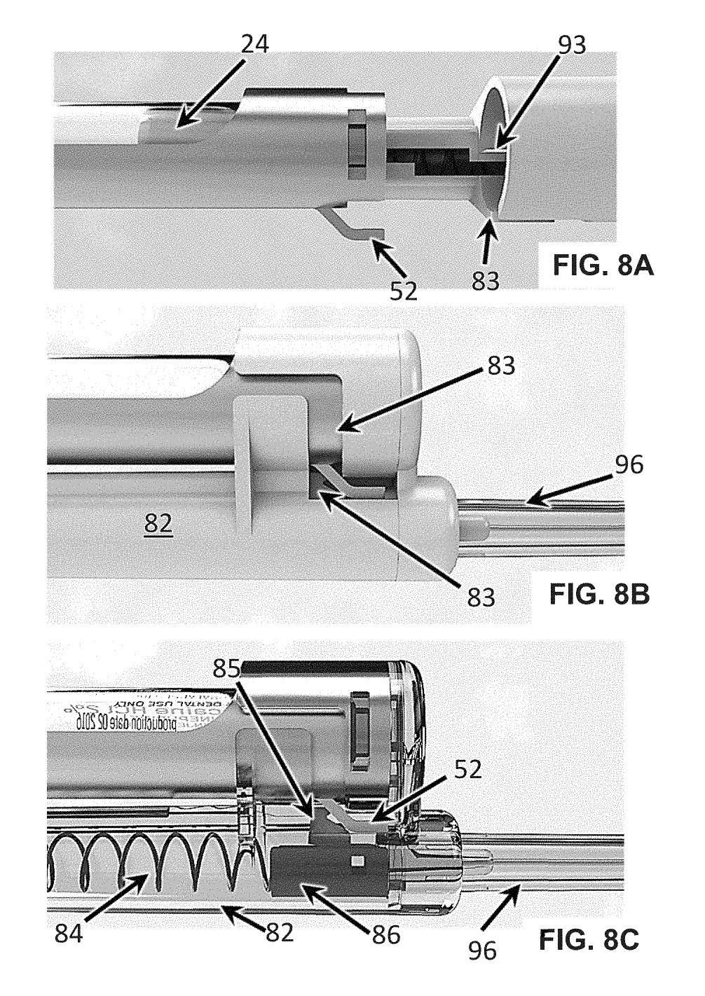

A hollow needle 98 projects from the distal end of housing 87, and is shown in FIGS. 3B and 3C safely surrounded by a sheath 96. In this first, extended, storage position sheath 96 reduces the likelihood of a user being punctured by the A bevel and sharp tip 99 of needle 98. By way of comparison, the sheath 96 is shown in a second, retracted, usage position in FIG. 9C, with tip 99 exposed.

Returning to FIGS. 3A, 3B, and 3C, it can be seen that receptacle 88 includes within it a retaining ring or cap 90 that is adapted and configured to receive within it an end of container 24. In one embodiment, container 24 is a carpule of known sizes to medical practitioners. However, in yet other embodiments needle assemble 80 can be adapted and configured to couple to an end of any type of container.

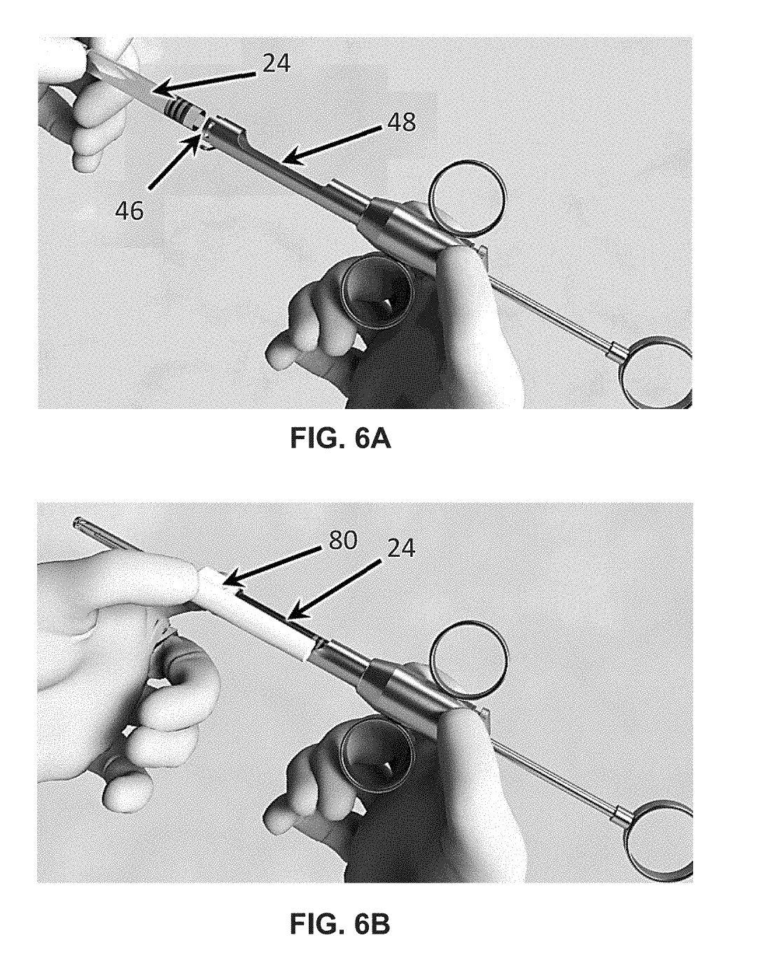

As best seen in FIGS. 3A and 7A, in one embodiment retaining ring 90 includes a plurality of ears or projections 91 that extend from the inner diameter of ring 90 toward the interior of the compartment 92 defined within ring 90. As will later be discussed with regards to FIGS. 7A, 7B, and 7C, projections 91 comprise one example of means for retaining container 24 within compartment 92.

The receptacle 88 of housing 87 further defines a cavity 94 that is adapted and configured to receive therein the distalmost end of syringe assembly 30. This cavity 94 generally surrounds ring 90 and compartment 92, and in one embodiment both the cavity and the compartment are generally cylindrical, and preferable co-axial. However, other embodiments of the present invention contemplate cavities that are separate from the compartments, and further non-co-axial.

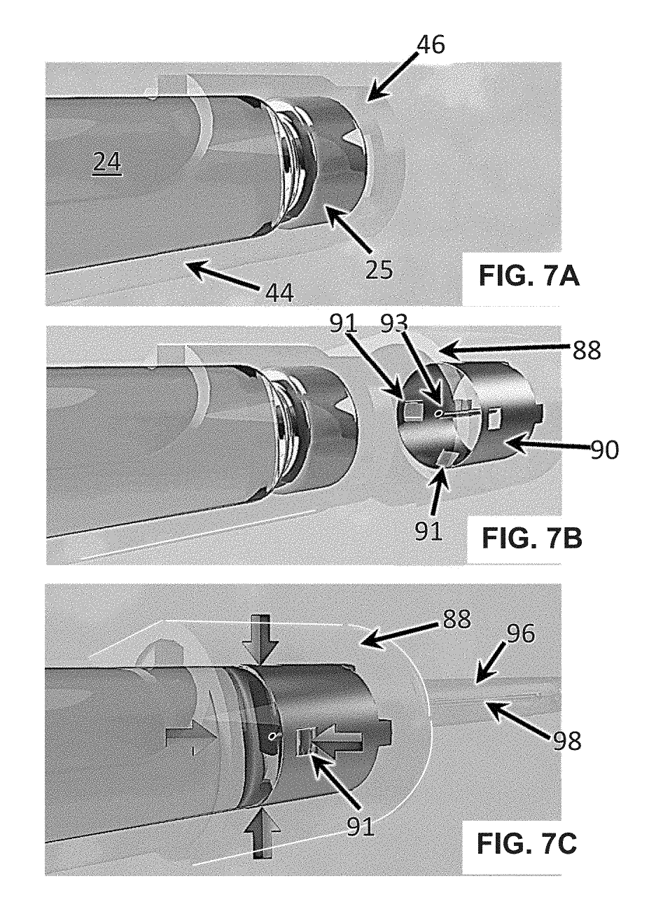

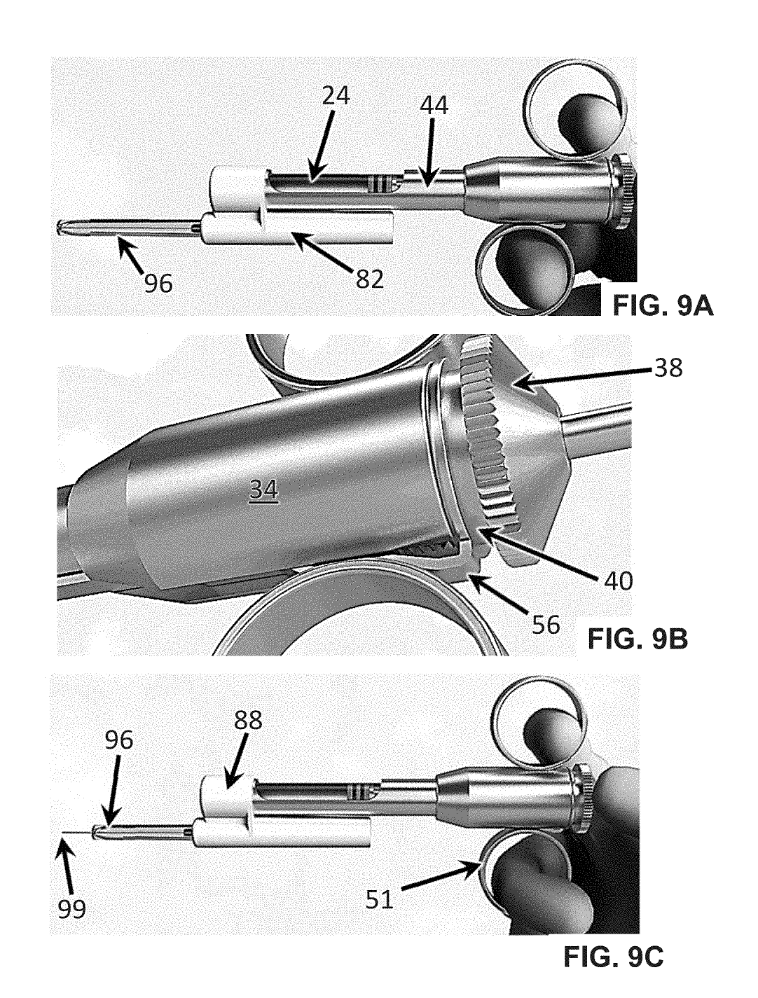

FIGS. 3B and 3C further show a slot 89 that is adapted and configured to receive within it the distalmost end of a slidable rail of syringe assembly 30, as will be discussed further in connection with FIGS. 8A and 8B. These figures further show a channel 83 that is generally parallel with the axis of needle 98. Referring briefly to FIG. 8C, it can be seen that a sliding block 86 located within housing 87 includes a topmost connector 85 that is located and slidable within channel 83. Block 86 (and therefore connector 85) are both attached to the proximal end of sheath 96. As will be discussed with regards to FIGS. 9C and 10B, the assembly of sheath 96, block 86, and connector 85 are slidable into housing 7, this grouping of components being guided by connector 85 within channel 83.

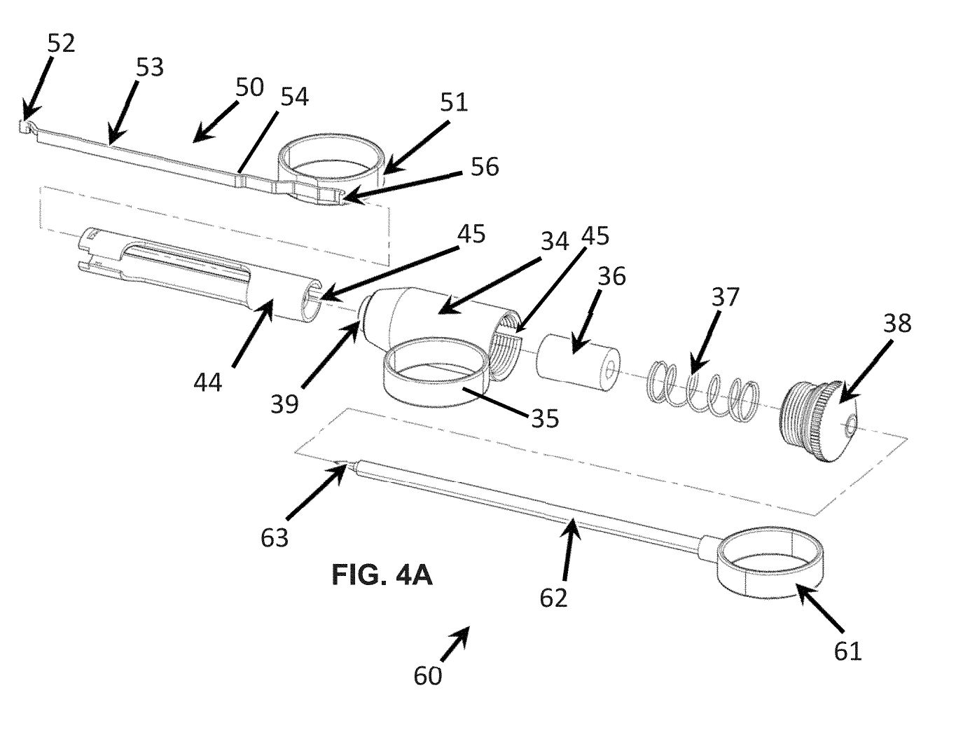

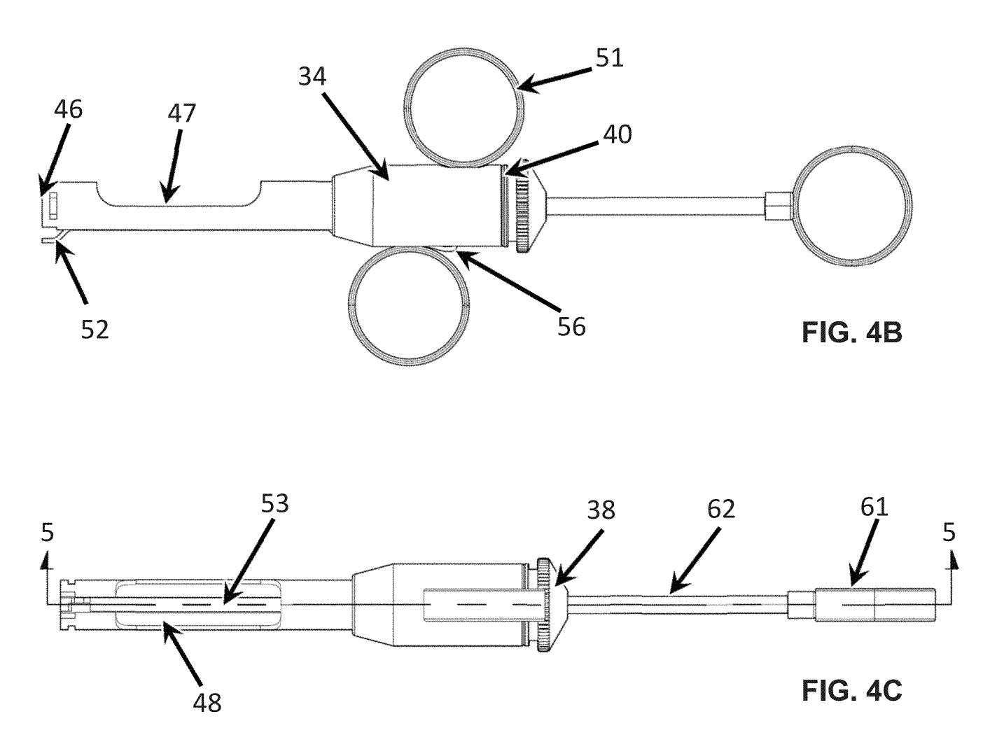

FIGS. 4A, 4B, and 4C show exploded and exterior views of syringe assembly 30. Body 34 receives within it a slidable spacer 36 that includes a proximal end in contact with a spring 37. Spring 37 is, in some embodiments, a coil spring, the other end of which is received within a pocket of a cap 38. The other end of spacer 36 is in contact with a shoulder 54 of rail 53, the operation of which will be discussed with regards to FIG. 5B and 5C.

The distalmost end of body 34 is attached to a container holder 44. Holder 44 includes an end received by guide 39 in the end of body 34. The other end of holder 44 is open and sized to receive therein an end of a container of liquid 24. Holder 44 further includes a channel 45 that both restrains and guides rail 53 of detent assembly 50.

Detent assembly 50 is axially slidable within channel 45, channel 45 extending axially along both body 34 and holder 44. Detent assembly 50 further includes a rail 53 which is configured to have a latch 56 on its proximal end, and a claw or projection 52 on its distal end. Rail 53 has a midsection between the claw 52 and a shoulder 54 that slides within channel 45. Shoulder 54 is adapted and configured to push against a surface of spacer 36. Spring 37 when compressed as shown in FIG. 5C applies a load to spacer 36, which subsequently applies an axial load to rail 53 by way of shoulder 54. This spring load assists in maintaining latch 56 within shoulder 40, as shown in FIG. 5C

Syringe assembly 30 also includes a pump 60. Pump 60 includes a shaft 62 that extends within and is supported by apertures in body 38, spacer 36, and guide 39. The distalmost end of shaft 62 includes a harpoon 63 that is adapted and configured to penetrate an end of a container 24 such as a carpule.

The other end of shaft 62 includes a finger hold 62, through which the user of assembly 20 applies a forward axial force to penetrate the carpule, and subsequently apply pressure to the liquid within the carpule. Assembly 20 preferably includes two other finger holds by which the user operates assembly 20. Detent assembly 50 includes a bottommost finger hold 51, and body 34 includes a topmost finger hold 35. Although what is shown and described are circular finger holds, it is understood that the present invention further contemplates any type of feature that permits a user of assembly 20 to hold body 34 in a position, move detent assembly 50 in a rearward direction, and move shaft 62 in a forward direction. In the embodiment shown in these figures, the injection assembly is adapted and configured to permit single handed operation for positioning the injection assembly, exposing the tip, and injecting the liquid. Further, these finger holds can be located in any suitable location consistent with these functions.

Holder 44 further includes a chamber 48 in which the container of liquid is held in place. A visualization port 47 permits the user to see the container during usage. In the embodiment shown, port 47 is an opened scallop of holder 44, but can be of any shape and further covered with a transparent material.

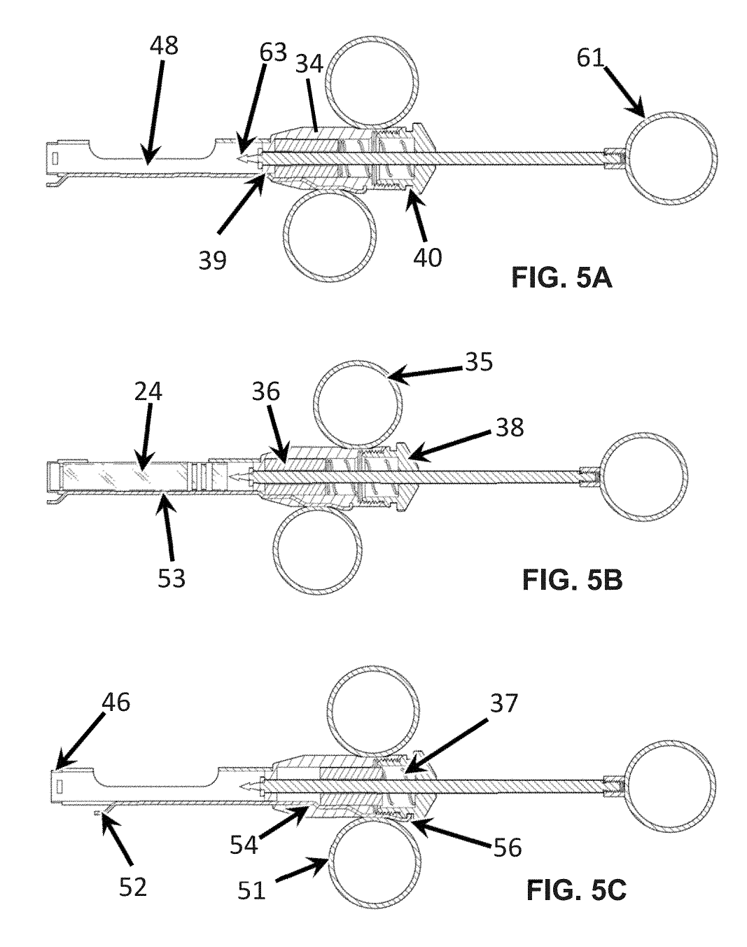

The operation of syringe assembly 30 is shown in FIGS. 5A, 5B, 5C, 6A, and 6B. FIG. 5A shows a cutaway of assembly 30 taken down the centerline. It can be seen that cap 38 is threadably received within body 34. In some embodiments, cap 38 is adapted and configured to include an inwardly directed shoulder 40 that cooperates with latch 56 to provide a detented, holding function of detent assembly 50 relative to body 34. Comparing FIGS. 5A and 5C, it can be seen that syringe assembly 30 has a first, storage state in which detent mechanism 50 is fully extended in a distal direction. FIG. 5A shows that in this first position, projection 52 is located generally proximate to entrance 46 of holder 44. Further, latch 56 is located at a home position along body 34.

FIG. 5C shows detent assembly 50 in a second, usage and temporary position in which the user has pulled rearwardly on finger hold 50, so as to slide rail 53 within channels 45. Projection 52 has moved to its proximal most location. Detent 56 is located within shoulder 40. Referring briefly to FIG. 9B, it can been seen that as the user pulls rearwardly (in a proximal direction) on finger hold 51 that rail 53 is able to elastically flex or bend outwardly over a ledge. FIG. 9B shows latch 56 in this outwardly bent position, immediately prior to being located in shoulder 40. The geometry and stiffness of rail 53 are chosen such that a typical user has sufficient strength to apply an expanding load between finger holds 35 and 51 so as to outwardly bend detent 56 to repeatedly move in and out of shoulder 40.

A detent mechanism is helpful in avoiding premature expelling of medicament. The force that pulls down on the trigger during injection may not be adequate if the entire liquid has not been completely expelled and the plunger has not bottomed out. In cases were the needle needs to be withdrawn, the operator can remove his thumb from the plunger loop, place it at the base of the syringe assembly so that adequate force can be applied to release the trigger, allowing it to slide up to its original location. That motion automatically allows the needle cover to slide back up upon removal of the needle covering it immediately.

FIGS. 6A and 5B show the insertion and positioning, respectively, of the container 24 within holder 44. FIG. 6A shows the user inserting an end of container 24 into an entrance 46 of holder 44. The user inserts the container 24 into holder 44 such that it is visible through port 47 within chamber 48, as shown in FIG. 5B.

FIGS. 6B, 7B, 7C, 8A, 8B, and 8C show the installation and coupling of a needle assembly 80 onto syringe assembly 30. FIG. 7A is a close-up, with exterior surfaces removed for improved visibility of the carpule 24 within holder 44 (similar to the view in FIG. 5B). Receptacle 88 of body 82 is then presented to the entrance 46 of holder 44, as best seen in FIGS. 6B and 7B. Needle assembly 80 is oriented relative to holder 44 such that claw 52 slides into the entrance of channel 83 (referring briefly to FIG. 8A). This coupling of needle assembly 80 to syringe assembly is further depicted in FIGS. 7B and 7C. It can be seen that the distal end 25 of carpule 24 is received within the inner compartment 92 of retaining ring 90, which happens either simultaneously, or after the reception of entrance 46 within cavity 94 of receptacle 88.

The alignment of entrance 46 with cavity 92 also aligns the axes of the B bevel 93 of a needle generally with the axis of carpule 24. The end 25 of the carpule is punctured by subsequent rearward axial movement of needle assembly 80, as shown externally in FIG. 6B. The internal components of this stage of coupling are shown in FIG. 7C. It can be seen that the ears 91 slide over the external surfaces of distal end 25.

Referring to FIG. 7B, it can be seen that the ears in one embodiment are prepared as a plurality (preferably 4) of circumferentially spaced apart, partial punch outs of retaining ring 90. These ears 91 are angled such that they elastically deflect outwardly, away from the outer diameter of end 25 of carpule 24 during insertion. Once ring 90 is fully seated on end 25 (as shown in FIG. 7C) these ears 91 each apply an inwardly directed radial load onto carpule 24. Preferably, these radial inward forces provide a snug feel to the coupling of carpule 24 within retaining ring 90. As shown in FIG. 7C, the B bevel 93 has fully penetrated end 24, and therefore establishes fluid communication within the hollow needle 98 to the A bevel, sharp tip 99.

After this coupling of the needle assembly 80 onto holder 44 has been accomplished, needle assembly 80 is then rotated to lock it into the usage position, as best seen in FIGS. 8B, 8C, and 9A. In comparing FIGS. 8A and 8B, it can be seen that claw 52, once it is received within the opened end of channel 83, then moves circumferentially within the channel as the operator rotates assembly 80 to the usage position. (This rotation accomplishing the change in position shown in comparing FIGS. 6B and 9A.) FIG. 8B shows a close up after this rotation has been accomplished, with projection 52 being located within channel 83, but now within a location in which projection 52 is in contact with connector 85 (best shown in FIG. 8C).

Once claw 52 is in contact with connector 85, the rearward (proximal direction) movement of rail 53 results in rearward movement of connector 85, block 86, and sheath 96. This rearward movement compresses spring 84 within housing 87. Referring to FIGS. 9A, 9B, and 9C, the rearward pulling by the user on finger hold 51 pulls sheath 96 in a rearward direction. The user also applies a radial outward force on finger hold 51, such that detent 56 is moved in a direction different than the sliding direction, and over a ledge of cap 38 and into shoulder area 40. This usage position is shown in FIG. 9C. Sheath 96 has been moved to a ready to use position, with A bevel 99 exposed.

FIGS. 10A and 10B depict usage of injection assembly 20 in a dental setting. The medical professional presents A bevel 99 to a location within the mouth of a patient selected for the introduction of an anesthetic located within a carpule 24. FIG. 10B shows the A bevel 99 located at the appropriate depth within the tissue of the patient. As shown in FIG. 10B, the needle 98 has been fully inserted, such that the tissue of the patient has contacted the distalmost end of the sheath 96. This contact between the tissue and the sheath is sufficient to further move the sheath within housing 87.

Referring briefly to FIG. 8C, it can be seen that the coupling of claw 52 and connector 85 is adapted and configured such that claw 52 can move connector 85 and sheath 96 in a rearward direction, but claw 52 does not prevent further rearward movement of sheath 96. After the medical professional has placed the A bevel 99 at the appropriate location, the professional provides an axial force to pump 60, causing harpoon 63 to apply pressure to the liquid within carpule 24, such that it flows from the B bevel to the A bevel and into the patient.

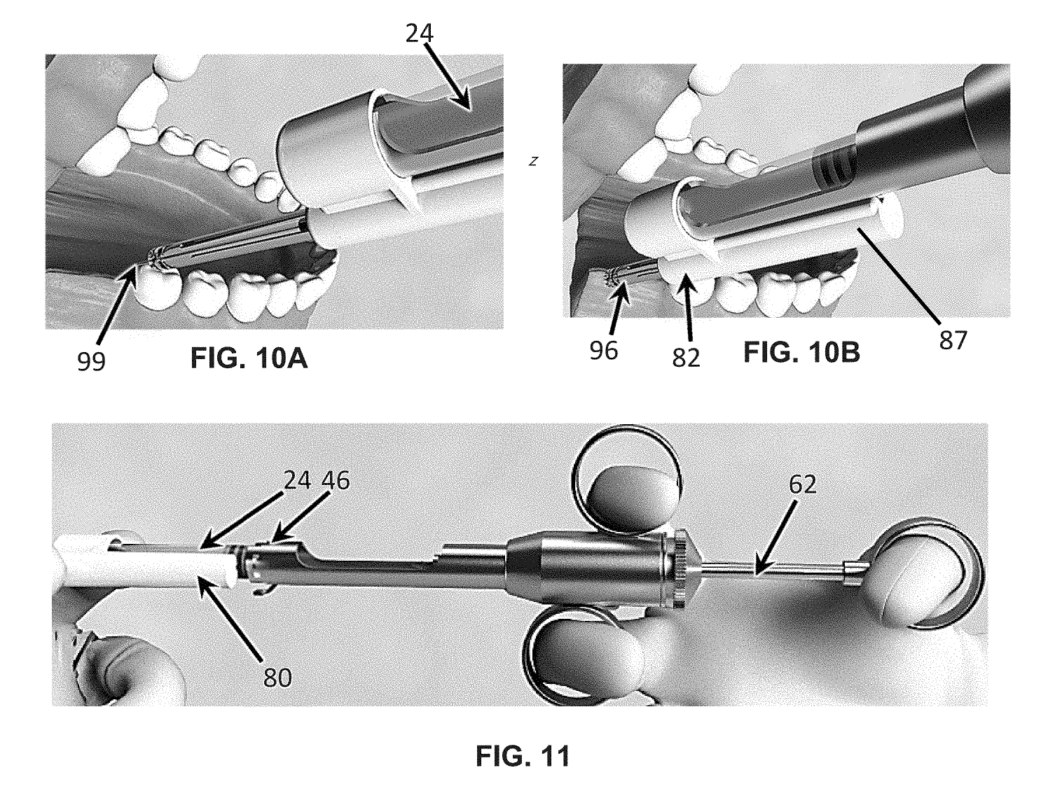

FIG. 11 illustrates the removal of needle assembly 80 from syringe 30. The user has rotated needle assembly 80, consistent with the pattern of the claw 52 within channel 83, as best seen in FIG. 8B. The needle assembly is rotated about 90 degrees and axially pulled from the entrance 46. As can be seen in FIG. 11, carpule 24 stays coupled to assembly 80 during removal. This retention of the carpule on assembly 80 is a result of the flexing interaction of ears 91 with the outer surface of end 25 of carpule 24, this interaction being one means of retention.

Referring to FIGS. 7B and 7C, FIG. 7B shows the inwardly ramped configuration of the ears. As carpule 24 is inserted into ring 90, the outer surface of the end 25 pushes downward on the ears 91, which elastically deflect out of the way. However, as shown in FIG. 7C, these ears maintain a radial force on the outer surface 25.

During removal, the configuration of the ears 91 is adapted and configured to provide an increase in the radial force, and therefore frictionally retain carpule 24 onto retaining ring 90. During removal, the outer surface 25 pulls rearward on the inward most, exposed, free edges of elastic ears 90. In pulling backward, this angular orientation of the ears provides a “Chinese handcuff” action, in which any attempt to separate carpule 24 from retaining ring 90 is met with increased resistance. The frictional force acting backward on the free exposed edge causes the ear to deflect even further toward the interior of compartment 92 and “bite” into the outer surface of end 25. There is a self-energizing effect, in which axial separation forces create increased radially inward directed forces. These radially inward forces in turn create an axial frictional force that is sufficient to maintain coupling of carpule 24 and ring 90, and overcome any other loads that would otherwise keep the carpule within the chamber 48. Thus, when the medical professional is done, the simple removal of needle assembly 80 also removes the carpule, and the combination of needle assembly and carpule can be safely disposed.

Various aspects of different embodiments of the present invention are expressed in paragraphs X1, X2, X3, and X4 as follows:

X1. One aspect of the present invention pertains to a method for dispensing liquid from a container with a syringe. The method preferably includes providing a syringe with compartment, a pump, and a sliding arm, and a separable needle assembly including a slidable sheath surrounding the sharp tip. The method preferably includes placing the container in the compartment, and attaching the needle assembly to the syringe. The method preferably includes engaging the sheath with the arm, sliding the arm, and exposing the sharp tip. The method preferably includes temporarily locking the arm in position with the sharp tip exposed.

X2. Another aspect of the present invention pertains to a method for dispensing liquid from a container with a syringe. The method preferably includes providing a syringe with compartment and a pump, and a separable needle assembly including a container-holding receptacle. The method preferably includes placing the container in the compartment, attaching the needle assembly to the syringe, and locating the container in the receptacle. The method preferably includes removing the needle assembly from the syringe; and retaining the container in the receptacle during and after the removing.

X3. Yet another aspect of the present invention pertains to an assembly for dispensing liquid from a container. The assembly preferably includes a syringe assembly having a distal end with an opening to a chamber, the opening being adapted and configured to receive a container of the liquid therethrough, the chamber being adapted and configured to hold the container, the syringe including a pump for expelling liquid from the container. The assembly preferably includes a separate needle assembly including a needle with a sharp tip, a sheath surrounding the sharp tip and slidable from a position surrounding the sharp tip to a position exposing the sharp tip, and a receptacle with an opening for receiving therein an end of the container, the needle assembly being adapted and configured to releasably couple to the syringe assembly. The assembly preferably includes means for retaining the container in the receptacle during removal of the needle assembly from the syringe assembly.

X4. Still another aspect of the present invention pertains to an assembly for dispensing liquid from a container. The assembly preferably includes a syringe assembly having a distal end with an opening to a chamber, the chamber being adapted and configured to hold the container, the syringe including a user pump for pressurizing the liquid and an arm slidable between a first position and a second position, the arm and the syringe being adapted and configured to releasably lock the arm on the syringe assembly in the second position. The assembly preferably includes a separate needle assembly including a needle with a sharp tip and a lumen, a sheath surrounding the sharp tip and slidable from a position surrounding the sharp tip to the second position exposing the sharp tip, a receptacle adapted and configured for receiving therein an end of the container, and a puncturing projection located within the receptacle, wherein coupling the receptacle to the end of the container punctures the end of container by the projection, and sliding the arm to the second position moves the sheath to the second position.

Yet other embodiments pertain to any of the previous statements X1, X2, X3, or X4 which are combined with one or more of the following other aspects. It is also understood that any of the aforementioned X paragraphs include listings of individual features that can be combined with individual features of other X paragraphs.

Wherein said locking is by interfering contact between the sliding finger hold and a non-sliding surface of the syringe

Which further comprises flexing the finger hold away from the syringe during said sliding.

Wherein said locking includes biasing the sliding finger hold in a direction opposite of the applied force.

Wherein said locking includes biasing the sliding finger hold in a direction toward the sheath surrounding the sharp tip.

Wherein said sliding is in a first direction, and said applying is in a different direction.

Wherein said retaining is by friction applied by the receptacle against the container.

Wherein said retaining increasing the friction applied by the receptacle against the container during said removing.

Wherein said retaining is by hooking the container with the receptacle.

Wherein application of an axial force to remove the container through the opening increases the frictional coupling of the container with said receptacle

Wherein said retaining means includes a projection adapted and configured to elastically deflect out of the way of the container during insertion of the container in the opening of said receptacle.

Wherein the projection is adapted and configured to elastically deflect into contact with the container during removal of the container from the opening of said receptacle.

Wherein said retaining means includes an ear projecting from the wall of the receptacle toward the container.

Wherein the ear is angled to permit easy coupling of said receptacle to the container and difficult removal of said receptacle from the container.

Wherein said retaining means includes a hook.

Which further comprises means for locking said arm in the second position.

Wherein said locking means includes a pair of external features that engage in the second position to interfere with sliding movement of said arm from the second position to the first position.

Wherein one of the features is a hook and the other of the features is a ledge or shoulder.

Wherein the external features disengage after said arm is flexed in a direction different than the direction of sliding.

Wherein said locking means includes a pair of complementary-shaped locking features, one of the pair being located on said arm and the other of the pair being located on said syringe assembly.

Wherein one of the features is a hook and the other of the features is a ledge or shoulder.

Wherein said locking means includes a male locking feature and a female locking feature, one of the male or female features being located on said arm and the other of the male or female features being located on said syringe assembly.

Wherein said locking means includes a pin and a hole, one of the pin or hole being located on said arm and the other of the pin or hole being located on said syringe assembly, the pin being receivable in the hole.

Wherein said locking means includes a latch that is movable in a direction different than the direction of sliding.

Wherein said arm is elastically flexible in a direction that is at least partly orthogonal to the direction in which said arm slides, said syringe assembly includes a receiving surface, said arm includes a latching surface, and said arm is locked to the second position by elastically flexing said arm to place the latching surface in contact with the receiving surface, wherein the receiving surface prevents the arm from sliding to the first position.

Wherein the receiving surface includes an outward projection, the latching surface includes a hook, and said arm is bent away from the syringe assembly to place the latching surface in contact with the receiving surface.

Wherein the outward projection is a ridge on said syringe assembly.

Which further comprises the means for self-energizing a frictional coupling of said receptacle to the container during separation of said needle assembly from said syringe assembly.

Wherein said needle has a centerline, said receptacle has a centerline, and the centerlines are not co-axial.

Wherein said needle assembly includes a spring to bias said sheath to surround said needle tip.

Wherein said arm includes a distal end having a projection and coupling of said needle assembly to said syringe assembly includes placing the projection in contact with said sheath.

Wherein the container is a carpule.

Wherein the projection includes a fluid passageway providing fluid communication between the container and the lumen.

While the inventions have been illustrated and described in detail in the drawings and foregoing description, the same is to be considered as illustrative and not restrictive in character, it being understood that only certain embodiments have been shown and described and that all changes and modifications that come within the spirit of the invention are desired to be protected.

What is claimed is:

1. A method for dispensing medicine from a container with a syringe, comprising:
providing a syringe with compartment, a pump, and a sliding finger hold, and a separable needle assembly including a slidable sheath surrounding the sharp tip;
placing the container in the compartment;
attaching the needle assembly to the syringe after said placing;
engaging the sheath with the finger hold;
sliding the finger hold and exposing the sharp tip;
locking the finger hold in position with the sharp tip exposed;
pumping medicine through the attached needle; and
applying a force with the finger hold to unlock from the position.

2. The method of claim 1 wherein said locking is by interfering contact between the sliding finger hold and a non-sliding surface of the syringe

3. The method of claim 1 which further comprises flexing the finger hold away from the syringe during said sliding.

4. The method of claim 1 wherein said locking includes biasing the sliding finger hold in a direction opposite of the applied force.

5. The method of claim 1 wherein said locking includes biasing the sliding finger hold in a direction toward the sheath surrounding the sharp tip.

6. The method of claim 1 wherein said sliding is in a first direction, and said applying is in a different direction.

7. A method for dispensing medicine from a container with a syringe, comprising:
providing a syringe with compartment and a pump, and a separable needle assembly including a container-holding receptacle;
placing the container in the compartment;
attaching the needle assembly to the syringe after said placing;
locating the container in the receptacle by said attaching;
pumping medicine through the attached needle;
removing the needle assembly from the syringe; and
retaining the container in the receptacle during and after said removing.

8. The method of claim 7 wherein said retaining is by friction applied by the receptacle against the container.

9. The method of claim 7 wherein said retaining increasing the friction applied by the receptacle against the container during said removing.

10. The method of claim 7 wherein said retaining is by hooking the container with the receptacle.

11. An assembly for dispensing liquid from a container, comprising:
a syringe assembly having a distal end with an opening to a chamber, the opening being adapted and configured to receive a container of the liquid therethrough, the chamber being adapted and configured to hold the container, said syringe including a finger-operated pump for expelling liquid from the container;
a separate needle assembly including a needle with a sharp tip, a sheath surrounding the sharp tip and slidable from a position surrounding the sharp tip to a position exposing the sharp tip, a receptacle with an opening for receiving therein an end of the container, said receptacle including a puncturing projection located within the receptacle, said needle assembly being adapted and configured to releasably couple to said syringe assembly; and
means for retaining the container in the receptacle during removal of said needle assembly from said syringe assembly;
wherein coupling said needle assembly to said syringe assembly punctures the end of the container by the projection.

12. The assembly of claim 11 wherein said retaining means is by friction.

13. The assembly of claim 12 wherein application of an axial force to remove the container through the opening increases the frictional coupling of the container with said receptacle.

14. The assembly of claim 11 wherein said retaining means includes a projection adapted and configured to elastically deflect out of the way of the container during insertion of the container in the opening of said receptacle.

15. The assembly of claim 14 wherein the projection is adapted and configured to elastically deflect into contact with the container during removal of the container from the opening of said receptacle.

16. The assembly of claim 11 wherein said retaining means includes an ear projecting from the wall of the receptacle toward the container.

17. The assembly of claim 11 wherein the ear is oriented to permit easy coupling of said receptacle to the container and difficult removal of said receptacle from the container.

18. The assembly of claim 11 wherein said retaining means includes a hook.

19. An assembly for dispensing liquid from a container, comprising:
a syringe assembly having a distal end with an opening to a chamber, the opening being adapted and configured to receive a container of the liquid therethrough, the chamber being adapted and configured to hold the container, said syringe including a finger-operated pump for pressurizing the liquid and an arm slidable between a first position and a second position, said arm and said syringe being adapted and configured to releasably lock said arm on said syringe assembly in the second position;
a separate needle assembly including a needle with a sharp tip and a lumen, a sheath surrounding the sharp tip and slidable from a position surrounding the sharp tip to the second position exposing the sharp tip, a receptacle adapted and configured for receiving therein an end of the container, and a puncturing projection located within the receptacle;
wherein coupling the receptacle to the end of the container punctures the end of container by the projection, and sliding the arm to the second position moves the sheath to the second position.

20. The assembly of claim 19 which further comprises means for locking said arm in the second position.

21. The assembly of claim 20 wherein said locking means includes a pair of external features that engage in the second position to interfere with sliding movement of said arm from the second position to the first position.

22. The assembly of claim 21 wherein one of the features is a hook and the other of the features is a ledge or shoulder.

23. The assembly of claim 21 wherein the external features disengage after said arm is flexed in a direction different than the direction of sliding.

24. The assembly of claim 20 wherein said locking means includes a pair of complementary-shaped locking features, one of the pair being located on said arm and the other of the pair being located on said syringe assembly.

25. The assembly of claim 24 wherein one of the features is a hook and the other of the features is a ledge or shoulder.

26. The assembly of claim 20 wherein said locking means includes a male locking feature and a female locking feature, one of the male or female features being located on said arm and the other of the male or female features being located on said syringe assembly.

27. The assembly of claim 20 wherein said locking means includes a pin and a hole, one of the pin or hole being located on said arm and the other of the pin or hole being located on said syringe assembly, the pin being receivable in the hole.

28. The assembly of claim 20 wherein said locking means includes a latch that is movable in a direction different than the direction of sliding.

29. The assembly of claim 19 wherein said arm is elastically bendable in a direction that is at least partly orthogonal to the direction in which said arm slides, said syringe assembly includes a receiving surface, said arm includes a latching surface, and said arm is locked to the second position by elastically bending said arm to place the latching surface in contact with the receiving surface, wherein the receiving surface prevents the arm from sliding to the first position.

30. The assembly of claim 29 wherein the receiving surface includes an outward projection, the latching surface includes a hook, and said arm is bent away from the syringe assembly to place the latching surface in contact with the receiving surface.

31. The assembly of claim 30 wherein the outward projection is a ridge on said syringe assembly.

32. The assembly of claim 19 wherein the means for self-energizing a frictional coupling of said receptacle to the container during separation of said needle assembly from said syringe assembly.

33. The assembly of claim 19 wherein said needle has a centerline, said receptacle has a centerline, and the centerlines are not co-axial.

34. The assembly of claim 19 wherein said needle assembly includes a spring to bias said sheath to surround said needle tip.

35. The assembly of claim 19 wherein said arm includes a distal end having a projection and coupling of said needle assembly to said syringe assembly includes placing the projection in contact with said sheath.

36. The assembly of claim 19 wherein the container is a carpule.

37. The assembly of claim 19 wherein the projection includes a fluid passageway providing fluid communication between the container and the lumen.
